(19) United States
(12) Patent Application Publication
(10) Pub. No.: US 2006/0281142 A1
(43) Pub. Date: Dec. 14, 2006

(54) COMBINED SAMPLE ENRICHMENT AND DISRUPTION

(75) Inventor: Frank R. Burns, Philadelphia, PA (US)

Correspondence Address: PATENT GROUP;ATTN: J. KENNETH JOUNG, DLA PIPER RUDNICK GRAY CARY US LLP, 203 N. LASALLE ST., SUITE 1900, CHICAGO, IL, 60601, US

(73) Assignee: E.I. DU PONT DE NEMOURS AND COMPANY, Wilmington, DE (US)

(21) Appl. No.: 11/379,098

(22) Filed: Apr. 18, 2006

Related U.S. Application Data
(60) Provisional application No. 60/672,922, filed on Apr. 19, 2005.

(51) Int. Cl. C12Q 1/04; C12M 1/34

(52) U.S. Cl. 435/34; 435/287.1

(57) ABSTRACT

A process of preparing a sample for analytical testing, comprising enriching and/or growing the sample in an enrichment/growth medium in a vessel; and disrupting the sample to release the intracellular contents of the sample, wherein the disruption is carried out in the same vessel as the enriching and/or growing. The sample is typically a biological sample, such as tissue, cells, microorganisms, or mixtures thereof.

This application claims the benefit of U.S. Provisional Application No. 60/672,922, filed Apr. 19, 2005, which is hereby incorporated by reference in its entirety.

### FIELD OF THE INVENTION

The field of invention relates to analytical testing of samples, and in particular, to preparation of samples such as biological samples for subsequent analysis of the intracellular components or fractions of the samples.

### BACKGROUND OF THE INVENTION

Analysis of biological samples can involve steps of growing and enriching a sample to increase the sample size, or to increase the number of target microorganisms in the sample up to detectable (or more readily detectable) levels, or to otherwise prepare the sample for subsequent analysis. For example, when testing food samples for the presence of pathogenic microorganisms, it is common to provide an aliquot of the food material in a suitable “enrichment medium” or “pre-enrichment medium”]in order to increase the population of the “target” microorganism (the particular microorganism whose presence as a contaminant is suspected) in the sample to be tested. As is known, the enrichment/pre-enrichment medium may include further agents which cause the target microorganism to be selectively enriched relative to other microorganisms which may be present. For disclosure of enrichment and pre-enrichment, attention is called to, e.g. U.S. Pat. No. 6,312.930, WO 98/20148; U.S. Pat. No. 5,843,669; U.S. Pat. No. 5,145,786; and EP 1 253 203 (published Oct. 30, 2002).

After enrichment, is it usually necessary to further prepare the sample for the intended analytical testing by disrupting cells. For example, if the aliquot contains cells or spores, and it is desired to analyze the nucleic acid contained within the cells or spores, the cells or spores must be disrupted to release their intracellular contents (including the nucleic acids). For example, pathogens in biological samples are sometimes detected by polymerase chain reaction (PCR). See, e.g., U.S. Pat. No. 6,312,930; WO 98.20148. To prepare a sample for PCR analysis the enriched sample is normally transferred from a first container, in which sample enrichment as carried out, to a second container in which cell disruption is performed to prepare the sample for PCR.

In the art, cell disruption is carried out by subjecting cells, spores or the like to forces which disrupt the cell walls, cell membrane, and other component structures of the cells to release the internal cell contents into solution. For example, in a technique referred to in the art as “bead beating,” a sample which contains cells or spores is agitated to cause disruptive contact between the cells and the disrupting elements sufficient to disrupt the cells and release their contents. See e.g. WO 98/11257; WO 2003008636. As a specific example, the sample solution, which may be previously enriched, can be introduced into a test tube containing glass beads. The combined solution containing the target microorganisms and the glass beads is then processed by application of force (e.g. centrifuge, vortex, etc.) which disrupts the cells. The phase which contains the released nucleic acids is then separated from the glass beads and other materials and is suitable for use in PCR analysis.

### SUMMARY OF THE INVENTION

It has now been found, surprisingly, that the enrichment or growth of a sample or a target microorganism in a sample may be carried out in the same vessel as a subsequent cell disruption process. It is not necessary to transfer the enriched sample from one vessel to another vessel between an enrichment step and a cell disruption step. Omission of this transfer step results in a less-labor intensive process and minimizes potential error or contamination which can occur during the transfer step.

In a first embodiment, the invention concerns a process for preparing a sample for analytical testing, which comprises:

a. enriching and/or growing the sample in an enrichment medium in a vessel; and

b. disrupting the sample to release the intracellular contents of the sample,

wherein the disruption is carried out in the same vessel as the enriching and/or growing.

In a more specific embodiment of the invention, the disruption of the sample is carried out by providing disrupting elements in the vessel and applying a force (or more than one force) to the vessel.

In a still further embodiment of the invention, a vessel is provided which contains both enrichment medium and disruptive elements.

### DESCRIPTION OF PREFERRED EMBODIMENTS

Each document cited herein is incorporated by reference in its entirety.

A sample in accordance with this invention may consist of any sample which comprises or contains tissue cells or microorganisms. Examples include biological tissue samples and food samples. A sample may consist of an aliquot of a larger sample.

Enrichment and/or growth of a sample in enrichment medium is well known to those in the art. Enrichment and/or growth as used herein will be understood as including enrichment, growth, pre-enrichment, selective enrichment, or any combination thereof. In applications such as food testing, it is desired to selectively enrich certain “target” microorganisms in the sample, such as certain strains of Salmonella and E. coli. Suitable medium for enrichment and/or growth are known in the art and are available commercially. Protocols for enrichment and/or growth are known in the art and are also disclosed in publicly-available FDA protocols.

Depending on the sample a target microorganism may be a bacterium, a fungus, or other type of microorganism. The target microorganism may exist in the sample in the form of a spore.

In accordance with the invention, the enrichment and/or growth is carried out in a vessel. A preferred type of vessel is a test tube, although other types of vessels such as beakers, flasks, jars, vials, ampules, microfuge tubes, etc. may be appropriate depending on the nature and size of the sample.

The disrupting elements are physical elements capable of disrupting tissue, cells, spores and the like to release their intracellular contents in the presence of an applied force. The elements may consist of particulate glass, plastic, metal or like material. Glass beads are currently preferred. The size of the disrupting elements may vary, but in the case of glass beads, a mean diameter of about 0.5 mm is currently preferred.

Disruption is carried out by application of force to the vessel such that the disrupting elements interact with the cells, tissue or spores to an extent that the intracellular contents of the sample and/or microorganisms in the sample is released. Force can be imparted to the vessel by centrifugation, sonication, stirring, vortexing, mixing, shaking or other agitation, optionally in combination with chemical disruption (e.g. use of detergent).

After disruption of the sample, the desired intracellular component(s) can be isolated from the disrupted sample solution for further analysis. Alternatively, a fraction of the disrupted sample solution which contains the desired intracellular components (e.g. the liquid phase) may be recovered for further analysis.

In a preferred embodiment, the disruptive elements are provided in the vessel together with the enrichment/growth medium, prior to addition of the sample. Thus, the pre-filled vessel (of sets of the vessels) can be stored for later use. All further processing as described can then be carried out in this same initial, pre-filled vessel. For example, to the vessel which contains both disruptive elements and enrichment/growth medium is added an aliquot of the sample, with dilution as may be appropriate. The enrichment/growth is allowed to proceed for a predetermined period of time under conditions to enrich the sample and/or target organisms in the sample, according to standard protocols known in the art. After the appropriate growth period, the vessel and its contents are subjected to disruptive forces to release the intracellular contents of the sample and/or target microorganisms, without transferring the enriched sample to a different vessel for the disruption. After disruption, the desired cellular components, phase or fraction of the disrupted mixture is recovered and can be analyzed.

It will be appreciated that, in many current analytical formats, multiple samples are processed together. The invention thus finds particular applicability in large scale, repetitive testing processes, where the omission of a separate transfer step has considerable practical value.

In a further embodiment, the invention lends itself to a “kit” format, wherein the kit comprises a vessel which contains a predetermined amount of a selected enrichment/growth medium and which further contains an effective amount of disruptive elements. The predetermined amount of a selected enrichment/growth medium is based on the particular type of sample to be enriched and the enrichment protocol associated therewith. The nature and quantity of disruptive elements should be effective to substantially disrupt the sample, or to substantially or entirely release the intracellular component being sought for further analysis, by application of the disruptive force to which the vessel is subjected.

The following example is provided as illustration and not as limitation.

### EXAMPLE 1

A 2.0 ml screw cap microcentrifuge tube as vessel was prepared to contain both a growth medium, in this case 1 mL of Potato Dextrose Broth with chloramphenicol and disrupting particles, in this case 1 gram of 0.5 mm zirconia/silica beads (BioSpec Products Inc., Bartlesville, Okla.). The tube was seeded with spores of an unknown species of mold isolated from a store bought food product. The tube was incubated for 2 days at 25 C to allow for growth of the organism. Following growth, the cells of the organism were disrupted by mechanical agitation by placing the same vessel in a Disruptor Genie (Scientific Industries Inc.) for three minutes. The liberated DNA was then amplified and detected by the polymerase chain reaction (PCR) using fungal specific primers. Sequencing of the PCR product and comparison of the sequence to the Genbank data base allowed the identification of the mold in the sample as Filobasidium globisporum.

1. A process of preparing a sample for analytical testing, which comprises:
a. enriching and/or growing the sample in an enrichment/growth medium in a vessel; and
b. disrupting the sample to release the intracellular contents of the sample,
wherein the disruption is carried out in the same vessel as the enriching and/or growing.

2. The process of claim 1, wherein the sample is a biological sample selected from the group consisting of tissue, cells, microorganisms, or mixtures thereof.

3. The process of claim 1, wherein the sample is a food sample which is suspected to contain a pathogenic microorganism.

4. The process of claim 1, wherein the vessel is a test tube or microfuge tube.

5. The process of claim 1, wherein the disruption is accomplished by providing disruptive elements in the vessel and subjecting the vessel and its contents to a force.

6. The process of claim 5, wherein the disrupting elements consist of particulate material.

7. The process of claim 6, wherein the particulate material is glass beads.

8. An article of manufacture comprising a vessel which contains a predetermined amount of growth/enrichment medium and an effective amount of disrupting elements.

9. The article of manufacture of claim 8, wherein the vessel is a test tube or microfuge tube and the disrupting elements are glass beads.
